(19) United States
(12) Patent Application Publication
(10) Pub. No.: US 2004/0002663 A1
(43) Pub. Date: Jan. 1, 2004

(54) Method and system for use in detecting a cancerous and precancerous pathology

(76) Inventor: William H. Reeves, Fort Lauderdale, FL (US)

Correspondence Address: A PATENT LAWYER CORP, PC, R WILLIAM GRAHAM, 22 S ST CLAIR ST, DAYTON, OH, 45402, US

(21) Appl. No.: 10/180,288

(22) Filed: Jun. 26, 2002

(51) Int. Cl. A61B 5/00

(52) U.S. Cl. 600/549

(57) ABSTRACT

A system detecting cancerous and precancerous pathologies includes a blood flow affecting compound introducible into an arterial and venous area of tissue in an area to be examined, a temperature sensor operably disposed on the area capable of sensing and generating a signal indicative of temperature sensed, and a computer-based device operably associated with the temperature sensor for receiving and manipulating the signal in a manner for generating a thermo-physiological data signal indicative of precancerous and cancerous conditions in the sensed area.

### BACKGROUND OF THE INVENTION

This invention is directed to a method and system for detection of cancerous and precancerous pathologies. More particularly to, but not by way of limitation, a method and system are provided for detection of cancerous and precancerous conditions in breasts, as well as established normal breast conditions.

There are several techniques known for detecting breast cancer. These techniques attempt to provide a physician with information based on either anatomical or physiological anomalies to enable the physician to make a determination as to the condition of the breast and both of these are limited. A primary concern with these techniques is that they avoid false positive testing when indicating an abnormality is present.

It is believed that tissue areas adjacent to carcinomas exhibit increased temperature from that exhibited contemporaneously by non-adjacent, non-cancerous tissue areas. The temperature of the cancer-affected areas can fluctuate several degrees Centigrade from normal tissue; these differences have been demonstrated while monitoring such areas for a 24-hour period (one circadian cycle). It is believed the detection of these temperature variations is indicative of cancers and enables detection of small cancers not otherwise detectable.

Despite the introduction of other detection techniques, mammography remains the standard method for testing for breast cancer. This is due in part to the low false positive ratio with mammography (erring on the side of not incorrectly advising the patient of a potential cancer). However, over thirty percent of patients with breast cancer malignancies are missed by radiologists. Mammography is, however, undesirable as it requires a radiological technique which passes ionizing radiation through the breast, which is per se invasive, to produce a radiograph which should report tumors as darkened areas. This method of detecting breast cancer is also limited by the age of the patient and condition of the tissue examined.

Other technology, as shown in U.S. Pat. Nos. 5,941,832 and 6,389,305, has shown the ability to detect significantly higher number of abnormal pathologies. It is thought that an abnormal temperature pattern associated with a tumor is a product of accelerated metabolism and numerous other factors, such as vaso-active substances and hormonal changes and increased regional blood flow caused by local angiogenesis, the detection of such abnormal patterns have led to unacceptable false positives. It still remains a belief that slight overall increase in the temperature of the surrounding tissue, for instance in localized areas of a woman's breast, is related to the vascular convection of heat that occurs as a result of capillary dilatation and the secondary increase in blood flow coupled with the higher temperature of the blood derived from the vascular bed and the possible vasodilator effect of catabolic products of a tumor metabolism. This alone is yet to produce an acceptable test for cancer detection. This is due in to the fact that such devices exhibit unacceptable false positive numbers.

Consequently, there remains a need to assist radiologists, surgeons and other physicians in detecting, diagnosing, successfully biopsing and operating on precancerous and cancerous conditions by providing improved detection methods and system with acceptable false positive ratios. It is believed that the method of employing a temperature sensing device is a preferred method of detection so long as the false positives can be eliminated.

### SUMMARY OF THE INVENTION

It is an object of the present invention to provide an improved method and device for detecting cancerous and precancerous pathologies.

It is yet another object of the present invention to provide a method and a device which employs an introduction of a blood flow affecting compound, which affect flow in an arterial and venous areas of tissue in an examined area which is concurrently monitored for temperature changes within the examined area thus enhancing temperature variations in the examined area and better aiding detection of cancerous and precancerous conditions.

The present invention is directed to a method and system for detection of cancerous and precancerous pathologies. The system includes a blood flow affecting compound introducible into an arterial and venous area of tissue in an area to be examined, a temperature sensing device operably disposed on the area capable of sensing and generating a signal indicative of temperature sensed, a computer-based device operably associated with the temperature sensing device for receiving and manipulating the signal in a manner for generating a thermo-physiological data signal indicative of precancerous and cancerous conditions in the sensed area.

The blood flow affecting compound can include one of a vasopressor and a vasoconstrictor, for example. The computer-based device can include non-algorithmic logic which utilizes prior pathological data in correspondence with the sensed signals to manipulate the signals and produce the thermo-physiological data signal. The non-algorithmic logic may include a neural network having predetermined solution space memory, the solution space memory including regions indicative of cancerous and non-cancerous conditions, wherein the thermo-physiological data produces the signals being projected into said regions.

The device further includes means operatively connecting the sensing means and the receiving and manipulating means, the connecting means includes means for receiving and storing the signals from the sensing means, means for controlling transmission of the signals from the sensing means to the storing means, and means for calibrating the sensing means for use in generating the thermo-physiological data.

Also, provided is a method for detection of cancerous and precancerous pathologies. The method includes introducing a blood flow affecting compound an arterial and venous area of tissue in an area to be examined, sensing temperature in the area, generating a signal indicative of temperature sensed, employing a computer-based device in a manner to be operably associated with the temperature sensor for receiving and manipulating the signal in a manner for generating a thermo-physiological data signal indicative of precancerous and cancerous conditions in the sensed area.

The present invention is more particularly described in the drawings and specification which follow. Other objects and advantages will be more readily apparent upon reading the following.

### BRIEF DESCRIPTION OF THE DRAWING

FIG. 1 shows a block diagram of the present invention.

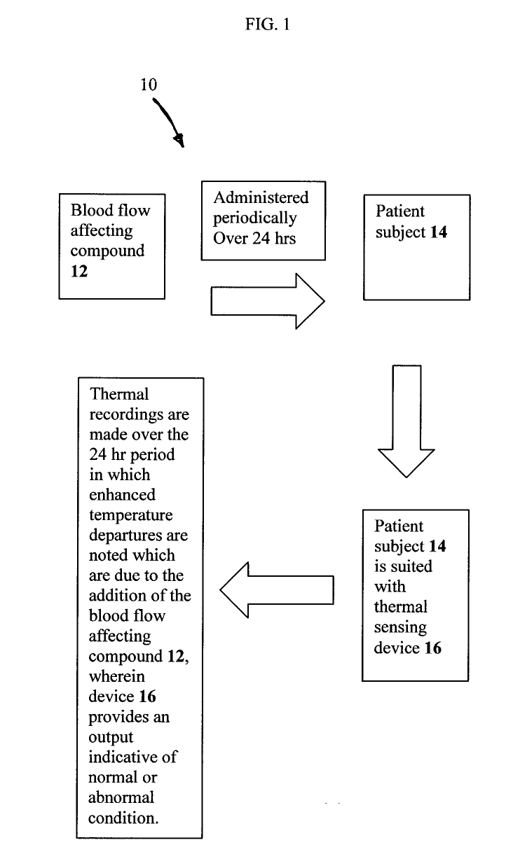

### DETAILED DESCRIPTION OF THE PREFERRED EMBODIMENT

Referring now to the invention and application thereof to breast cancer screening which currently has no adequate method of accurately discriminating between benign and cancerous lesions. Accordingly, the system present invention as hereinafter described will be referred to by the numeral 10.

The system 10 is predicated in part on a measurable thermal energy produced by lesions in the breast, either cancers or benign processes, which is the second order effect of two entirely different heat-producing mechanisms. Most malignant tumors produce new blood vessels in a process called neoangiogenesis.

These new vessels generally grow adjacent to the tumor, presumably to increase its nutrient supply and dispose of catabolic waste, etc. Studies of the microvascular patterns of capillaries in breast tissue from patients with intraducal carcinomas have demonstrated increased vascularity associated with many but not all intraductal carcinomas.

New vessel formation associated with intraductal carcinoma appears to be limited to the region of the basement membrane in ducts with intraductal carcinoma. Evidence of angiogenesis is found in 80% of intraductal carcinomas consisting of a ring of neovascularity completely or partially encircling the affected duct. It has been found that comedocarcinomas had a complete ring of neovascularity, whereas non-comedo lesions tend to have a partial ring or no neovascularity.

These tumor blood vessels differ from normal vessels in many respects. Their irregular size, tortuosity, abnormal wall structure, heterogenicity, and leakiness can all be regarded as bizarre hallmarks of a propensity to break all the rules for the construction of normal vessels. Most tumor vessels have irregular profiles, abnormal branching patterns, and do not conform to usual classification of arterioles, capillaries, and venules. The periocyte coat and basement membrane of the tumor vessels are also abnormal.

Tumor vessels with a normal smooth muscle coat are rare, and even large-caliber vessels may have capillary-like wall structure. The lack of smooth muscle renders these newly formed vessels unreceptive to control by epinephrine, thus, the deficiency in cholinergic receptiveness produces a constant blood flow in and around the tumor. The resultant tumor temperature has a less circadian temperature variation than is evident in healthy tissue. As will be recognized, this becomes a very important aspect in the amplifying the operation of the system 10.

An example of the use of the invention is revealed in the detection of breast cancer. Benign changes often accompanied with breast pain prior to the premenstruum are most often the result of an endocrine disturbance in women with short menstrual cycles of 21 to 24 days. The pain, at first only presents in the premenstruum, becoming more severe until it persists throughout the cycle. The affected breast is usually well developed.

A swollen granular zone of increasing density may frequently be felt in the upper lateral quadrant more often than in other quadrants. Benign changes are often bilateral, but definitive masses are not always felt. Biopsies prove to be more dense and fibrous that normal tissue.

Excessive estrogen, so many in the medical community postulate, causes breast tissue to accumulate fluid. As breast volume increases, excess pressure is placed on sensory nerve endings, causing pain. Then a homeostatic reflex response (increase in regional blood to remove the irritants) occurs, the thermal energy output is increased by the increased blood flow.

On the other hand, it is thought that developing breast cancer is a summation of a large number of mutations which occur over many years, each with its particular phenotypes. The progression of benign morphologies can be seen in a multifocal manner when examining a pre-menopausal cancer mastectomy in the remaining tissue away from the cancer.

Angiogenesis in preneoplastic and neoplastic lesions found at http://www.slip.net/˜mcdavis/database/angio11.htm citing Angiogenesis as a marker of preneoplastic lesions of the human breast. Cancer 1978; 41:239-244 Jensen H M, Chen I, DeVault M R, Lewis A E, Jensen and her colleagues studied the frequency and distribution of so-called “atypical lobules.” Angiogenesis at http://ccm.ucdavis.edu/tgmouse/JENSEN-MAMM2000/ANGIO-1/ANGIO-1.HTML citing Angiogenesis induced by normal human breast tissue: a probable marker for precancer. Science 1982; 218: 293-29; Brem S S, Jensen H M, Gullino P M. . These modules of excessive epithelium are seen with a dissecting microscope and correspond to hyperplasia in routine histopathic studies. Jensen found these structures more common in mammary tissue ipsilateral and contralateral to the cancers.

The present invention thus presumes that cancer does not initiate in a normal mammary epithelium but rather after a series of epithelial proliferations. Further, these physiological changes can be associated with an “abnormal temporal phenotype,” which is related to an abnormal menstrual metabolic cycle within the breast, each having its own thermal signature. These signatures can be measured and potentially exploited to assess risk in a particular breast.

It is discovered by the present invention that the undesirable false positives which are found using thermal sensing can be reduced to acceptable levels while providing significantly higher cancerous detection rates. This is achieved by introducing a blood flow affecting compound 12 into the body of the patient/subject 14. The compound is target specific to affect blood flow in normal epithelium tissue, but not in the abnormal epithelium proliferated tissue.

The compound, for example, can be a vasopressor (which is an agent that stimulates contraction of the muscular tissue of the capillaries and arteries) or vasoconstrictor (which can be a chemical substance which causes vasoconstriction, or the narrowing of blood vessels so that less blood is able to flow through at a time), and preferably works on specific normal tissue receptors causing contraction of the tissue and thereby restricting blood flow in the vessels therein. The vasopressor or vasoconstrictor compound chosen should not affect receptors in the abnormal tissue and thus does not constrict the vessels therein and blood flow therethrough. The present invention can employ several types of vasopressors to accomplish the intended function. Examples of several vasopressors are:

1. Ana-Kit Anaphylaxis Emergency Treatment Kit:

Epinephrine Injection

Chemically, epinephrine is 1-(3,4-dihydroxyphenl)-2(methylaminoethanol with the following structure:

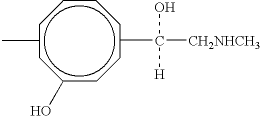

2. ProAmatine Tablets:

Chemical Names:

(USAN: Midodrine Hydrochloride): (1)Acetamide, 2-amino-N-[2-(2,5-dimethoxyphenyl)-2-hydroxyethyl]-monohydrochloride, (±)−; (2) (±)−2-amino-N-(β-hydroxy-2,5-dimethoxyphenethyl)acetamide monohydrochloride BAN, INN, JAN: Midodrine

3. Vasozyl Injection:

Methoxamine hydrochloride.

### EXAMPLE CASE

Midodrine, a vasoconstrictor, was used to carry out the efficacy of the invention. Midodrine has been studied in three principal controlled trials, one of three-weeks duration and two of one to two days duration.

All studies were randomized, double-blind, and parallel-design trials in patients with orthostatic hypotension of any etiology and supine-to-standing fall of systolic blood pressure of at least 15 mmHg accompanied by at least moderate dizziness/lightheadedness. Patients with pre-existing, sustained supine hypertension above 180/110 mmHg were routinely excluded.

In a three-week study in 170 patients (most previously untreated with midodrine) the midodrine-treated patients (10 mg t.i.d.,—with the last dose not later than 6 P.M.) had significantly higher (by about 20 mmHg) one-minute standing systolic pressure one hour after dosing. Blood pressures were not measured at other times for the entire three weeks.

After week one, midodrine-treated patients had small improvements in dizziness/light headedness/unsteadiness scores and global evaluations, but these effects were made difficult to interpret by a high, early dropout rate (about 25% vs. 5% on placebo). Supine and sitting blood pressure rose 16/8 and 20/10 mmHg, respectively. In a two-day study, after open-label midodrine, known midodrine responders received midodrine 10 mg or placebo at 0, 3, and, 6 hours. One-minute standing systolic blood pressures were increased one hour after each dose by about 15 mmHg and three hours after each dose by about 12 mmHg; three-minute standing pressures were increased also at one, but not 3, hours after dosing.

There were increases in standing time seen intermittently one hour after dosing, but not at three hours. In a one-day, dose-response trial, single doses of 0, 2.5, 10, and 20 mg of midodrine were given to twenty-five patients. The 10 and 20-mg doses produced increases in standing one-minute systolic pressure of about 30 mmHg at one hour; the increase was sustained in part for two hours after 10 mg and four hours after 20 mg. Supine systolic pressure was greater than 200 mmHg in 22% of patients on 10 mg and 45% of patients on 20 mg; and elevated pressures often lasted six hours or more.

It is found ProAmatine™ (Midodrine) forms an active metabolite, desglymidodrine, (1059) that is an alph1-agonist, and exerts its actions via activation of the alpha-adrenergic receptors of the arteriolar and venous vasculature, producing an increase in vascular tone and elevation of blood pressure. Desglymidodrine does not stimulate cardiac beta-adrenergic receptors.

Desglymidodrine diffuses poorly across the blood-brain barrier and is, therefore, not associated with effects on the central nervous system. Administration of ProAmatine results in a rise in standing, sitting, and supine systolic and diastolic blood pressure in patients with orthostatic hypotension of various etiologies. Standing systolic blood pressure is elevated by approximately 15 to 30 mmHg at one hour after a 10-mg dose of midodrine, with some effect persisting for two to three hours. ProAmatine has no clinically significant effect on standing or supine pulse rates in patients with autonomic failure.

Fifteen women volunteers selected for the purposes of this study have the following characteristics: five women judged to have normal breasts after professional examination and mammography, five women judged to have benign disease after professional examination and mammography and pathology, and five women judged to have cancer after professional examination and mammography.

Each is suited to wear the thermal sensing device 16 as is described in U.S. Pat. No. 5,941,832, the specification of which is hereby incorporated by reference, and in a manner as described therein for forty-eight hours and perform their normal, daily duties. After the first twenty -four hours, the patient is administered a 10 mg dose of Midodrine to achieve an effective level of blood flow affecting compound 12 and which level is maintained with a periodic additional dosage of 10 mg every six hours over a twenty-four hour period of data collection. This level can fluctuate depending upon the particular individual's weight and physical condition.

Data is collected and analyzed with device 16, which can be over the circadian period and further by aid of employing a neural network which can be used to determine the abnormality or normality of the tissue. The effect of the vasoconstrictor on the vascularity of the breast pronounces the difference in temperature exhibited (or the thermal energy) by the breast lesions and thus more effectively segregate into normal, suspicious, and abnormal categories the examined tissue. The invention thus lowers false positive ratio obtained by the device 16.

The above described embodiment is set forth by way of example and is not for the purpose of limiting the present invention. It will be readily apparent to those skilled in the art that obvious modifications, derivations and variations can be made to the embodiment without departing from the scope of the invention. Accordingly, the claims appended hereto should be read in their full scope including any such modifications, derivations and variations.

What is claimed is:

1. A system for detection of cancerous and precancerous pathologies, which includes:
a blood flow affecting compound introducible into an arterial and venous area of tissue in an area to be examined;
a temperature sensing device operably disposed on the area capable of sensing and generating a signal indicative of temperature sensed; and
a computer-based device operably associated with the temperature sensing device for receiving and manipulating the signal in a manner for generating a thermo-physiological data signal indicative of precancerous and cancerous conditions in the sensed area.

2. The system for detection of cancerous and precancerous pathologies of claim 1, wherein said blood flow affecting compound includes one of a vasopressor and vasoconstrictor.

3. The system for detection of cancerous and precancerous pathologies of claim 1, wherein said blood flow affecting compound includes at least one of epinephrine, midodrine hydrochloride, and methoxamine hydrochloride.

4. A method for detection of cancerous and precancerous pathologies in a tissue area of a subject, which includes the steps of:
introducing a blood flow affecting compound an arterial and venous area of tissue in an area to be examined within the subject;
sensing temperature in said area;
generating a signal indicative of temperature sensed;
employing a computer-based device in a manner to be operably associated with the temperature sensor for receiving and manipulating the signal in a manner for generating a thermo-physiological data signal indicative of precancerous and cancerous conditions in the sensed area.
